(19) United States
(12) Patent Application Publication
(10) Pub. No.: US 2018/0369431 A1
(43) Pub. Date: Dec. 27, 2018

(54) Ex vivo labeled myeloid derived suppressor cells (MDSC) for cancer imaging

(71) Applicant: Mehryar Khalili Garakani, Danville, CA (US)

(72) Inventor: Mehryar Khalili Garakani, Danville, CA (US)

(21) Appl. No.: 15/629,747

(22) Filed: Jun. 22, 2017

(51) Int. Cl. A61K 51/12

(52) U.S. Cl. CPC A61K 51/1203

(57) ABSTRACT

The present invention relates to myeloid derived suppressor cells (MDSCs) and using these cells in conjunction with nuclear medicine imaging methods to image cancer tumors. The MDSC cells are separated from blood and either radiolabeled with nuclear medicine agents or pretargeting agents and reinjected in patient. The cells preferentially concentrate in tumors allowing imaging of cancer tumors. The imaging technique can be used to image tumors for diagnosis and/or assessment of treatment response for cancer patients undergoing cancer treatments.

### CROSS-REFERENCE TO RELATED APPLICATIONS

This application claims the benefit of provisional patent application Ser. No. 62/352,936 filed 21 Jun. 2016 by the present inventor, which is incorporated by reference to its entirety.

### TECHNICAL FIELD

The present invention relates to nuclear medicine imaging, and more particularly to imaging of cancer tumors.

### BACKGROUND

The main clinical method of imaging cancer used today relies on 2-deoxy-2[F-18] fluoro-D-glucose positron emission tomography, also known as FOG-PET. This relies on increased metabolism of cancer cells compared to normal cells to allow increased concentration of F18 radionuclide in cancer cells, which allows Positron Emission Tomography (PET) imaging devices to form a 3D image of cancer tumors. The FDG-PET image allows identifying the extent of disease (staging) and hence impact the patient treatment decisions.

Concentration of FDG in cancer cells relies on the fact that they typically have higher sugar metabolism rates than regular cells (i.e., are “highly glycolytic”). However, this is not always true. Cancers that are not highly glycolytic do not image well on FDG-PET, as it can be difficult to distinguish them from regular cells in an FDG-PET image due to their lack of higher sugar metabolic rates.

As a result of preferential localization of MDSCs to tumor sites, this invention allows radionuclide levels to be higher in tumor cells than in most normal tissues (except possibly spleen and liver). This can substantially improve the Signal-to-Noise Ratio (SNR) associated with the nuclear medicine images of many body areas, and provides a better image contrast.

### NONPATENT LITERATURE DOCUMENTS

Bronte, Vincenzo, et al. “Recommendations for myeloid-derived suppressor cell nomenclature and characterization standards.” Nature communications 7 (2016).
Rege, Sheila, et al. “Use of positron emission tomography with fluorodeoxyglucose in patients with extracranial head and neck cancers.” Cancer 73.12 (1994): 3047-3058.
Guze, Barry H., et al. “Indium-111 White Blood Cell Scans: Sensitivity, Specificity, Accuracy, and Normal Patterns of Distribution.” Clinical nuclear medicine 15.1 (1990): 8-10.

### Definitions

The term nuclear medicine agent refers to a molecule that contains a radioactive nuclide suitable for imaging using positron emission tomography, single photon emission computed tomography, or scintigraphy.

The term pretargeting agent, in the context of this invention, refers to a molecule that binds to surface of MDSC and additionally has a second binding site for a nuclear medicine agent.

The term MDSC refers to myeloid derived suppressor cells, which are a group of heterogeneous cells of myeloid origin.

The term PMN-MDSC refers to polymorphonuclear myeloid derived suppressor cells. One way to identify PMN-MDSCs from mice is to use the following molecular signature: CD11b⁺ Ly6C^{low} Ly6G⁺. One way to identify PMN-MDSCs from humans is to use the following molecular signature: CD14⁻ CD11b⁺ CD15⁺ (or CD66b⁺).

The term M-MDSC refers to monocytic myeloid derived suppressor cells. These are a group of MDSCs that have attributes similar to the monocyte lineage. One way to identify M-MDSCs from mice is to use the following molecular signature: CD11b⁺ Ly6C^{hi} Ly6G⁻. One way to identify M-MDSCs from humans is to use the following molecular signature: CD14⁺ CD11b⁺ HLA-DR^{low/-} CD15⁻.

### SUMMARY OF THE EMBODIMENTS

A first embodiment includes making a composition comprising of a nuclear medicine imaging agent attached to or uptaken by MDSCs. The said composition is used in a method of imaging a cancer tumor in an organism, which comprises of:
injecting the said composition into the organism,
waiting for a period for myeloid derived suppressor cells to localize to the cancer tumor,
imaging the organism using a standard nuclear medicine imaging technique.

A second embodiment includes making a composition comprising of a nuclear medicine pretargeting agent attached to MDSCs. The said composition is used in a method of imaging a cancer tumor in an organism, which comprises of:
injecting the said pretargeted myeloid derived suppressor cell composition into the organism,
waiting for a period for myeloid derived suppressor cells to localize to the cancer tumor,
injecting a nuclear medicine imaging agent that binds to the said pre-targeting agent,
imaging the organism using a standard nuclear medicine imaging technique.

### BRIEF DESCRIPTION OF THE DRAWINGS

FIG. 1 shows the steps used in the first embodiment of this invention.

FIG. 2 shows the steps used in the second embodiment of this invention.

FIG. 3 shows some example radionuclides that can be incorporated into the imaging agents used with this invention.

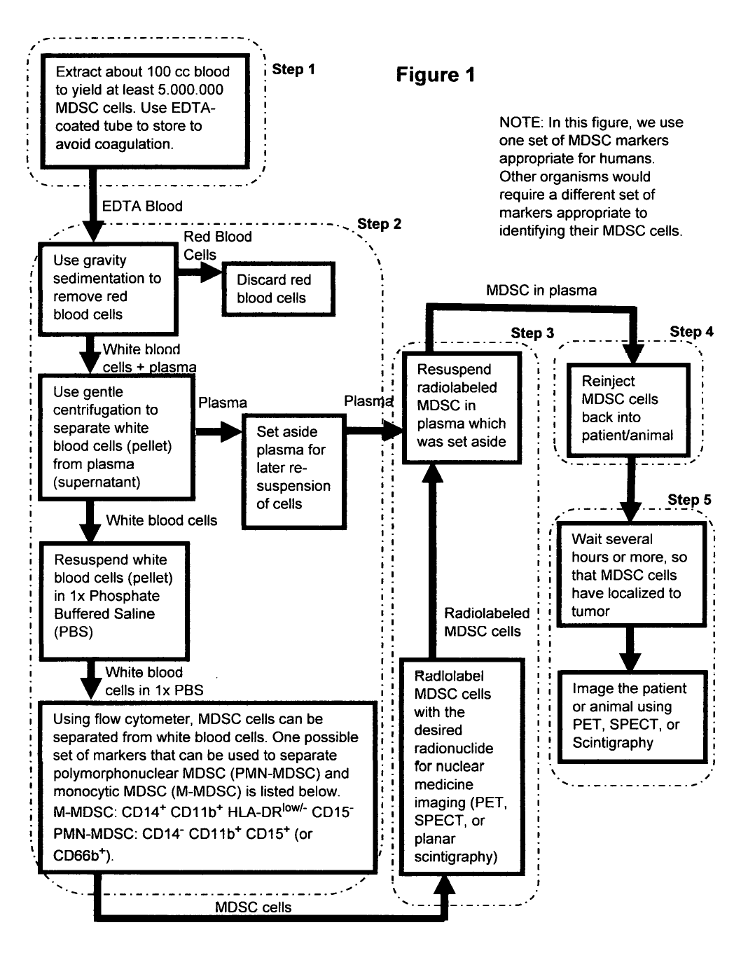

### DETAILED DESCRIPTION—FIG. 1—FIRST EMBODIMENT

As stated above, the main clinical method of imaging cancer used today relies on 2-deoxy-2[F-18] fluoro-D-glucose positron emission tomography, also known as FDG-PET. This relies on increased metabolism of cancer cells compared to normal cells to allow increased concentration of F18 radionuclide in cancer cells, which allows Positron Emission Tomography (PET) imaging devices to form a 3D image of cancer tumors. The FDG-PET images allow identifying the extent of disease (staging) and hence impact the patient treatment decisions.

Concentration of FDG in cancer cells relies on the fact that they typically have higher sugar metabolism rates than regular cells (i.e., are highly glycolytic). However, this is not always true. Cancers that are not highly glycolytic do not image well on FDG-PET, as it can be difficult to distinguish them from regular cells in an FDG-PET image due to their lack of higher sugar metabolic rates. The invention claimed here solves this problem.

The invention described here uses a different principal to image cancer tissues, which does not rely on their potentially higher metabolic rates. Instead we rely on the fact that cancer tumors are frequently immunosuppressed.

The immunosuppressed tumor microenvironment is frequently associated with the presence of a large number of myeloid derived suppressor cells (MDSC), which play a key role in suppressing immune system in the tumor environment and allowing cancer tumors to evade immune response.

The MDSC concentration is frequently increased in the blood circulation of cancer patients, These MDSCs can be separated from the patient's blood (i.e., harvested) and radiolabeled with a radioactive molecule ex vivo. The radiolabeled MDSCs can then be re-injected back into the patients, at which time they will migrate preferentially to the sites of the primary and metastatic tumors. This allows concentrating imaging radionuclide agents in the tumor tissues and provides a better method to image cancer using standard imaging equipment such as gamma camera, single photon emission computed tomography (SPECT), or positron emission tomography (PET).

This invention is an improvement on what currently exists. The described method does not rely on high sugar metabolism rate of cancer tissues and hence can be effective in imaging cancers that do not have high sugar metabolism making them unsuitable for FDG-PET imaging.

The composition consisting of MDSCs plus nuclear medicine imaging agent can be used to image cancer tumors in humans as well as other mammals (including companion animals) having cancer.

The term patient in this document refers to either a human, or any other mammal, including companion animals under veterinary care.

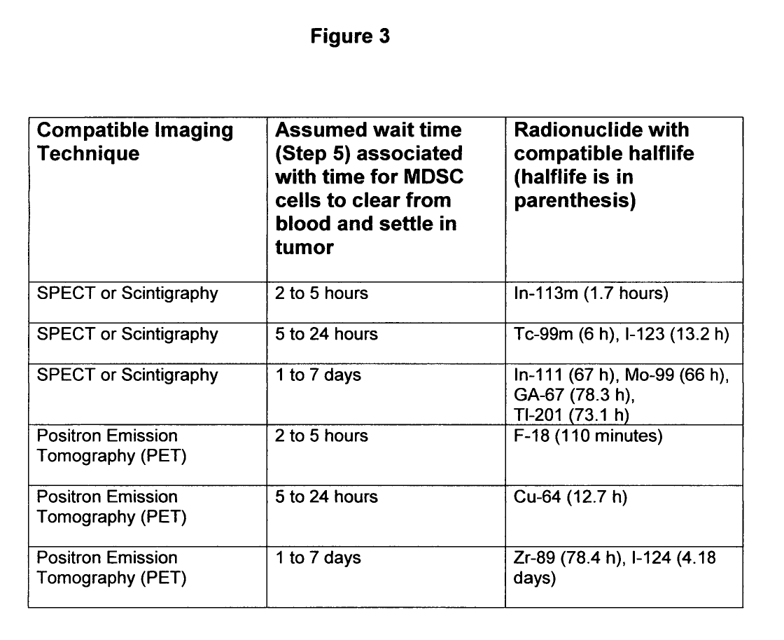

This first embodiment, which is shown in FIG. 1, includes the following methods:
1. Withdraw blood from the patient
2. Separate MDSC cells using any of the number of techniques known in the arts. These could be PMN-MDSCs or monocytic MDSCs or both
3. Compose a radiolabeled MDSC composition comprising of separated MDSC cells and a nuclear medicine agent bounded to MDSC cell membrane using one or more of hydrogen bonding, van der Waals, ionic, covalent, or hydrophobic interaction or uptaken into MDSC cells. The nuclear medicine agent carries a radionuclide that is suitable for nuclear medicine imaging, with some examples shown in FIG. 3, for illustration. The composition can be constructed by attaching nuclear medicine imaging agent to MDSC cell membrane or uptaken by MDSC cells
4. Reinject the radiolabeled MDSCs back into the patient
5. Image patient using standard nuclear medicine imaging

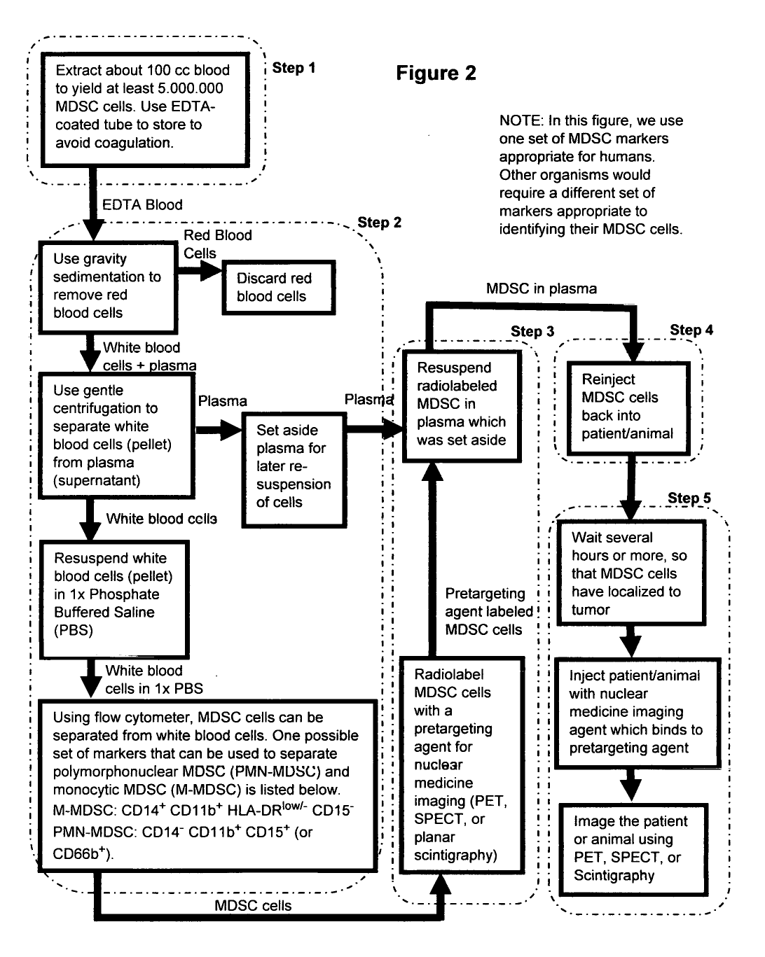

### Alternative Embodiment—FIG. 2

In an alternative embodiment in Step 3, a composition comprised of a pretargeting agent attached to MDSC cell membrane is used. The pretargeting agent is a non-radioactive agent that can bind to MDSCs using one or more of hydrogen bonding, van der Waals, ionic, covalent, or hydrophobic interactions and that has a binding site for a nuclear medicine agent that can be attached later In Step 5 as shown in FIG. 2.

1. A method of imaging a cancer tumor in a human or animal organism, which comprises of:
Labeling the organism's myeloid derived suppressor cells with a nuclear medicine imaging agent ex vivo,
reinjecting the said labeled myeloid derived suppressor cells into the organism,
waiting for a period for myeloid derived suppressor cells to localize to the cancer tumor,
imaging the organism using a standard nuclear medicine imaging technique.

2. The method of claim 1, wherein only polymorphonuclear myeloid derived suppressor cells are used.

3. The method of claim 1, wherein only monocytic myeloid derived suppressor cells are used.

4. The method of claim 1, wherein the said labeling is achieved by binding the nuclear medicine imaging agent to the cell membrane of myeloid derived suppressor cells.

5. The method of claim 1, wherein the said labeling is achieved by having the nuclear medicine agent uptaken into the myeloid derived suppressor cells.

6. The method of claim 1, wherein the said nuclear medicine imaging technique is positron emission tomography.

7. The method of claim 1, wherein the said nuclear medicine imaging technique is single-photon emission computed tomography.

8. The method of claim 1, wherein the said nuclear medicine imaging technique is scintigraphy.

9. A method of imaging a cancer tumor in a human or animal organism, which comprises of:
Labeling the organism's myeloid derived suppressor cells with a nuclear medicine pre-targeting agent ex vivo which binds to the outside of the cell membrane of myeloid derived suppressor cells,
reinjecting the said labeled myeloid derived suppressor cells into the organism,
waiting for a period for myeloid derived suppressor cells to localize to the cancer tumor,
injecting a nuclear medicine imaging agent that binds to the said pre-targeting agent,
imaging the organism using a standard nuclear medicine imaging technique.

10. The method of claim 9, wherein only polymorphonuclear myeloid derived suppressor cells are used.

11. The method of claim 9, wherein only monocytic myeloid derived suppressor cells are used.

12. The method of claim 9, wherein the said nuclear medicine imaging technique is positron emission tomography.

13. The method of claim 9, wherein the said nuclear medicine imaging technique is single-photon emission computed tomography.

14. The method of claim 9, wherein the said nuclear medicine imaging technique is scintigraphy.

15. A myeloid derived suppressor cell composition comprising one or more molecules of either a nuclear medicine pre-targeting agent or a nuclear medicine imaging agent attached to the outside of plasma membrane of myeloid derived suppressor cells of a human or animal organism.

16. The composition of claim 15, wherein the said pre-targeting agent molecules are bounded to the outside of plasma membrane of the said myeloid derived suppressor cell using any one or a combination of hydrogen bonds, covalent bonds, ionic bonds, van der Waals or hydrophobic interactions.

17. The composition of claim 15, comprising of the said nuclear medicine agent molecules bounded to the outside of plasma membrane of the said myeloid derived suppressor cell using any one or a combination of hydrogen bonds, covalent bonds, ionic bonds, van der Waals or hydrophobic interactions.

18. The composition of claim 15, comprising of the said nuclear medicine agent molecules up-taken by the said myeloid derived suppressor cells
